(19) United States
(12) Plant Patent Application Publication
(10) Pub. No.: US 2004/0107469 P1
(43) Pub. Date: Jun. 3, 2004

(54) Floribunda rose plant named 'Scrivo'

(76) Inventor: Leonard W. Scrivens, Kidderminster (GB)

Correspondence Address: CHRISTIE, PARKER & HALE, LLP, P.O. BOX 7068, PASADENA, CA, 91109-7068, US

(21) Appl. No.: 10/306,925

(22) Filed: Nov. 29, 2002

(51) Int. Cl. A01H 5/00

(52) U.S. Cl. PLT/143

(57) ABSTRACT

A new variety of Floribunda rose suitable garden decoration, having flowers of creamy white coloration.

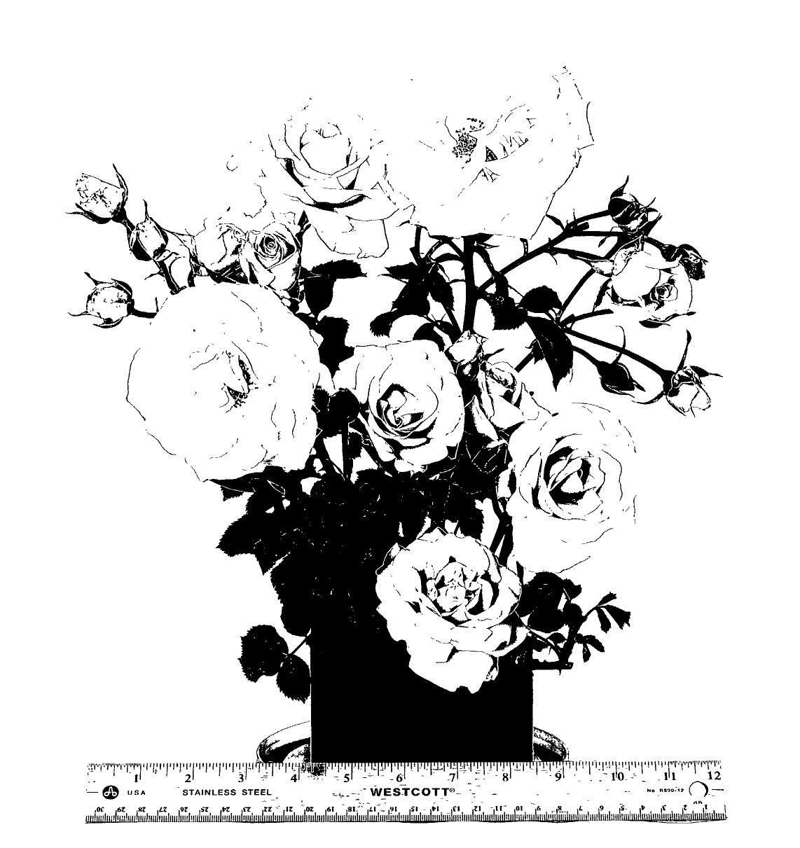

### BACKGROUND OF THE INVENTION

This invention relates to a new and distinct variety of Floribunda Rose. The varietal denomination of the new variety is ‘SCRivo’. It has undisseminated seedlings of my creation as its seed and pollen parents.

### SUMMARY OF THE INVENTION

Among the features which distinguish the new variety from other presently available and commercial rose cultivars known to the inventor are the following combination of characteristics: its excellent black spot resistance, its very wide stipules with very long straight points, its very short styles, its red suffusion on the rachis and on the stipules of the young leaves and its broad range of peduncle length. The plant has a bushy to slightly spreading growing habit, suitable for outdoor garden decoration.

Asexual reproduction of the new variety by budding as performed in Kern County and Upland, Calif., shows that the foregoing and other distinguishing characteristics come true to form and are established and transmitted through succeeding asexual propagations. ‘SCRivo’ may be asexually propagated by cuttings, budding and grafting.

### COMPARISON WITH PARENTS

The new rose may be distinguished from its seed parent, an undisseminated seedlings of my creation by the following combination of characteristics: whereas ‘SCRivo’ bears large (about 7.4 to about 11.2 cm. in diameter) semi-double (about 20 to 24 petals) flowers of creamy white coloration, the seed parent bears significantly larger (about 10.5 to about 14.3 cm. in diameter) flowers of yellow washed orange coloration and of significantly higher petalage (about 30 to 45 petals). The new variety is a floribunda with a bushy to slightly spreading growing habit, whereas the seed parent is a hybrid tea with a significantly taller more upright mature growth habit.

The new variety may be distinguished from its pollen parent, an undisseminated seedlings of my creation by the following combination of characteristics: whereas ‘SCRivo’ bears flowers of creamy white coloration with semi-double petalage (about 20 to 24 petals), the pollen parent bears flowers of pale pink and yellow coloration and of significantly lower petalage (about 5 petals). The new variety has a bushy to slightly spreading medium height growing habit, whereas the pollen parent has a significantly shorter and more compact growing habit.

### BRIEF DESCRIPTION OF ILLUSTRATION

The accompanying photograph illustrates the new variety and shows the flowering thereof from bud to full bloom depicted in color as nearly correct as it is possible to make in a color illustration of the character. Throughout this specification, color references and/or values are based upon the Colour Chart of The Royal Horticultural Society (1966) except where common terms of color definition are employed.

### DESCRIPTION OF THE NEW VARIETY

The following description is of 3 to 4 year-old rose plants of the new variety grown outdoors in Upland, Calif. in the month of October. Phenotypic expression may vary with environmental, cultural and climatic conditions, as well as differences in conditions of light and soil.

### FLOWER

The new variety sometimes bears its flowers singly, usually in clusters of three to four or more per stem. Flowers may be borne in irregular somewhat pyramidal clusters on strong short stems (about 24 to about 57 cm.). Outdoors, the plant blooms very abundantly and nearly continuously during the growing season. The flowers have a slight tea fragrance.

### BUD

The peduncle is about 2.4 to about 5.9 cm. in length, of somewhat heavy caliper (about 0.2 to about 0.4 cm. in diameter), and usually erect and stiff. It is almost entirely smooth, with few stipitate glands. Peduncle color is between 144B and 146D often heavily suffused, especially on the side exposed to the sun, with between 183C and 187B.

Before the calyx breaks, the bud is about 1.5 to about 1.9 cm. in diameter at the widest point, about 1.6 to about 2.2 cm. in length, and ovoid to moderately pointed in shape. The surface of the bud bears few foliaceous appendages, usually with slender entire to somewhat cut foliaceous parts extending beyond the tip of the bud about ¼ or more of its length. Bud color is between 144A and 146B sometimes lightly suffused, especially on the side exposed to the sun, with between 187B and 183C.

The sepals are about 2.6 to about 3.4 cm. in length and about 0.8 to about 1.2 cm. in width at the widest point. The outer surface color of the sepal is between 144A and 146B sometimes lightly suffused, especially on the side exposed to the sun, with between 187B and 183C. The inner surface color of the sepal is between 138B and 137C and covered with fine wooly tomentum; sepal margins are lined with some stipitate glands and hairs.

As the petals open (after the calyx breaks), the bud is about 1.8 to about 2.3 cm. in diameter at the widest point, about 2.2 to about 2.7 cm. in length, and very ovoid to somewhat pointed in form. The color of the under surfaces of the newly opened petals is between 38B and 49C. At the point where the petal attaches, there is a moderately large zone of between 10C and 8C. On the upper surfaces of the newly opened petals, at the point where the petal attaches, there is a moderately large zone of between 12A and 9B that gradually suffused to between 14D and 12D near the lower center of the petal, continuing to suffuse to between 38A and 36A toward the petal edge.

### BLOOM

When fully open, the bloom ranges from about 7.4 to about 11.2 cm. in diameter. Petalage is double with about 20 to 24 petals and about 0 to 2 petaloids irregularly arranged. When partially open, the bloom form is moderately high centered to cupped to somewhat globular, and the petals are moderately tightly spiraled to cupped with petal edges slightly reflexed outward. When fully open, the bloom form is more cupped, and the petals are somewhat loosely cupped with petal edges moderately reflexed outward.

### PETALS

The substance of the petals is somewhat heavy and of medium thickness, with upper surfaces moderately shiny and under surfaces more matte to slightly shiny. The outer petals are nearly round in shape with rounded apices. The inner petals are moderately obovate to somewhat oval in shape with rounded apices. The petals are about 2.9 to about 4.3 cm. in length and about 2.7 to about 4.5 cm. in width at the widest point. Petal margins are entire.

### NEWLY OPENED FLOWER

The under surface color of the outer petals is between 55D and 49C. At the point where the petal attaches, there is a moderately large zone of between 10C and 9D. On the upper surfaces of the outer petals, at the point where the petal attaches, there is a moderately large zone of near 12B that gradually suffused to between 8D and 10D near the lower center of the petal, continuing to suffuse to between 49C and 36D toward the petal edge.

The under surface color of the intermediate and inner petals is between 39C and 36C. On the upper surfaces of the intermediate and inner petals, at the point where the petal attaches, there is a moderately large zone of near 12B that gradually suffused to between 12D and 13D near the lower center of the petal, continuing to suffuse to between 27B and 27D toward the petal edge.

The general tonality of the newly opened flower is between 12D and 13D near the lower center of the petal, gradually suffusing to between 27B and 27D toward the edge of the petal.

### THREE-DAY-OLD FLOWER

The under surface color of the outer and inner petals is between 56B and 56D. At the point where the petal attaches, there is a moderately small zone of between 8D and 11D. The upper surface color of the outer and inner petals is between 56D and 155D. At the point where the petal attaches, there is a moderately large zone of between 10C and 8C.

The general tonality of the three-day-old flower is between 56D and 155D.

On the spent bloom, the petals usually drop off cleanly.

In October in Upland, Calif., blooms on the bush growing outdoors generally last about four to five or more days. Cut roses from plants grown outdoors and kept at normal indoor living temperatures generally last about four to five or more days.

### MALE REPRODUCTIVE ORGANS

Stamens are many in number (average about 111) and are arranged regularly about the pistils; a few are mixed with petaloids. The filaments are of variable length (about 0.3 to about 1.0 cm.), most with anthers. Filaments are between 7A and 9B in color. The anthers are of medium size for the class and all open approximately at the same time. Anther color is between 20A and 14C when immature and between 165A and 167A at maturity. Pollen is very abundant.

### FEMALE REPRODUCTIVE ORGANS

Pistils vary in number (average about 63). The styles are moderately even, very short in length (about 0.2 to about 0.4 cm.), somewhat thin in caliper, and moderately bunched. Stigma color is near 10B. Style color is near 2D. Ovaries are usually all enclosed in the calyx.

Hips have not been observed on this variety when grown in Upland, Calif.

### FOLIAGE

The compound leaves are usually comprised of three to seven or more leaflets and are borne abundantly. The Seven-leaflet leaves are about 7.2 to about 10.1 cm. in length and about 5.1 to about 7.6 cm. in width at the widest point, moderately leathery in texture, and semi-glossy to somewhat glossy in finish. The terminal leaflets are about 2.7 to about 4.2 cm. in length and about 2.2 to about 3.6 cm. in width at the widest point, shaped ovate to somewhat oval with moderately acute apices and rounded bases. Their margins are usually simply serrate.

The upper surface color of the mature leaf is darker than between 137A and 147A. The under surface color of the mature leaf is between 147B and 138B. The upper surface color of the young leaf is between 137A and 147B, sometimes lightly suffused toward the edge of the leaves with between 187B and 183B. The under surface color of the young leaf is between 147B and 138B, sometimes lightly suffused with between 187B and 183B.

The rachis is average in caliper and somewhat rough. The upper side is deeply grooved with many small hairs and some stipitate glands on the edges of the grooves. The under side of the rachis is somewhat rough with few small prickles. The rachis color is between 145B and 144D often heavily suffused on the young leaves with between 187B and 183B.

The stipules are about 0.9 to about 1.4 cm. in length and very wide (about 0.5 to about 0.8 cm.) with very long straight points that usually turn out at an angle of more than 45 degrees and sometimes recurve toward the stem. The stipules color is between 137C and 145B often heavily suffused on the young leaves with between 187B and 183B.

The petiole is about 0.5 to about 1.3 cm. in length and about 0.1 to about 0.2 cm in diameter at the widest point. The petiole color is between 145B and 144D often heavily suffused on the young leaves with between 187B and 183B.

The plant displays an average degree of resistance to powdery mildew and rust as compared to other commercial varieties grown under comparable conditions in Upland, Calif. The plant's winter hardiness and drought/heat tolerance are yet to be determined.

### GROWTH

The plant has a bushy to slightly spreading medium height growing habit (about 105 to about 125 cm. in height and about 88 to about 105 cm. spread at the widest point), with full branching. It displays moderately vigorous growth and the canes are of somewhat light caliper for the class.

The color of the major stems is near 146C. They bear several large prickles that are about 0.7 to about 1.1 cm. in length. The large prickles are almost straight and angled slightly downward with a moderately long broad to oval base; prickle color is between 165B and 165C. The major stem bears many small prickles of similar shape and coloration.

The color of the branches is between 144B and 148A sometimes lightly suffused with between 183C and 187C. They bear some large prickles that are about 0.4 to about 0.7 cm. in length and of similar shape to the prickles on the major stem. Prickle color is near 160B sometimes lightly suffused with between 183D and 187D. The branches bear few small prickles of similar shape and coloration.

The color of the new shoots is between 144B and 146C sometimes lightly suffused with between 183C and 187C. They bear few large prickles which are of similar size and shape to the large prickles on the branches; prickle color is near 160B often lightly suffused with between 183D and 187D. The shoots bear few small prickles of similar shape and coloration.

I claim:

1. A new and distinct Floribunda rose plant of the variety substantially as described and illustrated herein.
